(19) United States
(12) Patent Application Publication
(10) Pub. No.: US 2023/0168010 A1
(43) Pub. Date: Jun. 1, 2023

(54) TEMPERATURE CONTROL DEVICE

(71) Applicant: TOYOTA JIDOSHA KABUSHIKI KAISHA, Toyota-shi Aichi-ken (JP)

(72) Inventors: Keiko IIDA, Nagoya-shi Aichi-ken (JP); Tomoya YAMAGUCHI, Seto-shi Aichi-ken (JP)

(73) Assignee: TOYOTA JIDOSHA KABUSHIKI KAISHA, Toyota-shi Aichi-ken (JP)

(21) Appl. No.: 17/989,127

(22) Filed: Nov. 17, 2022

(30) Foreign Application Priority Data
Nov. 30, 2021 (JP) 2021-194269

(51) Int. Cl. F25B 21/02; A61F 7/00

(52) U.S. Cl. CPC F25B 21/02; A61F 7/007; A61F 2007/0075; F25B 2321/0212

(57) ABSTRACT

A temperature control device has a temperature acquisition unit that acquires information indicating a temperature around a user and a control unit that controls a Peltier element worn by the user to cool or warm the user based on the temperature. The control unit causes the Peltier element to warm the user when the temperature is higher than or equal to a first threshold and causes the Peltier element to cool the user when the temperature is lower than a second threshold.

### CROSS REFERENCE TO RELATED APPLICATION

This application claims priority to Japanese Patent Application No. 2021-194269 filed on Nov. 30, 2021, which is incorporated herein by reference in its entirety including the specification, claims, drawings, and abstract.

### TECHNICAL FIELD

The present disclosure relates to a device for controlling a cooling and warming element that is worn by the user to cool or warm the user.

### BACKGROUND

JP 2010-19433 A describes a small air conditioner for cooling and warming the human body locally by means of a Peltier element.

Although this small air conditioner described in JP 2010-19433 A can cool or warm the body, it merely cools or warms the body locally, and it is not always able to effectively control the temperature to be perceived by the user.

An object of the present disclosure is to effectively control the temperature to be perceived by a user wearing a cooling and warming element.

### CITATION LIST

### Patent Literature

Patent Document 1: JP 2010-19433 A

### SUMMARY

An aspect of the present disclosure is a temperature control device that includes an acquisition unit that acquires information indicating a temperature around a user, a cooling and warming element that is worn by the user to cool or warm the user, and a control unit that controls the cooling and warming element based on the temperature acquired by the acquisition unit, and in this temperature control device, the control unit causes the cooling and warming element to warm the user when the temperature is higher than or equal to a first threshold and causes the cooling and warming element to cool the user when the temperature is lower than a second threshold that is lower than the first threshold.

According to the above configuration, the user is warmed when the temperature around the user is higher than or equal to the first threshold. The user is cooled when the temperature around the user is lower than the second threshold. For example, the user is warmed when it is hot around the user. The user is cooled when it is cold around the user. Heating the user causes heat dissipation from the user's body. Cooling the user causes heat production in the user's body. The temperature perceived by the user is thus controlled by using the user's own heat dissipation and heat production.

The cooling and warming element may be placed on the back of the user's head.

The cooling and warming element may be placed at a position corresponding to the central nerve system of the user. This makes it possible to effectively cool or warm the central nerve system of the user, thereby allowing the user's own heat production or heat dissipation to occur in an effective manner.

The control unit switches on and off the cooling and warming element repeatedly, and when a temperature of a user-facing surface of the cooling and warming element does not fall below a third threshold while the cooling and warming element performs cooling, or when the temperature of the user-facing surface of the cooling and warming element does not rise above a fourth threshold while the cooling and warming element performs warming, the control unit may make the length of time during which the cooling and warming element is off longer than in other cases.

The cooling and warming element is a Peltier element, for example.

According to the present disclosure, it is possible to effectively control the temperature to be perceived by the user wearing the cooling and warming element.

### BRIEF DESCRIPTION OF DRAWINGS

An embodiment of the present disclosure will be described based on the following figures, wherein:

FIG. 1 is a block diagram illustrating a configuration of a temperature control device according to an embodiment;

FIG. 2 is a side view illustrating a Peltier element worn on a user's head;

FIG. 3 is a back view illustrating the Peltier element worn on the user's head;

FIG. 4 is a diagram illustrating the principle to raise a temperature to be perceived;

FIG. 5 is a diagram illustrating the principle to raise a temperature to be perceived;

FIG. 6 is a diagram illustrating the principle to lower a temperature to be perceived;

FIG. 7 is a flowchart illustrating operations of the temperature control device;

FIG. 8 is a graph illustrating changes in surface temperature of the Peltier element over time; and

FIG. 9 is a flowchart illustrating operations according to a modified example.

### DESCRIPTION OF EMBODIMENTS

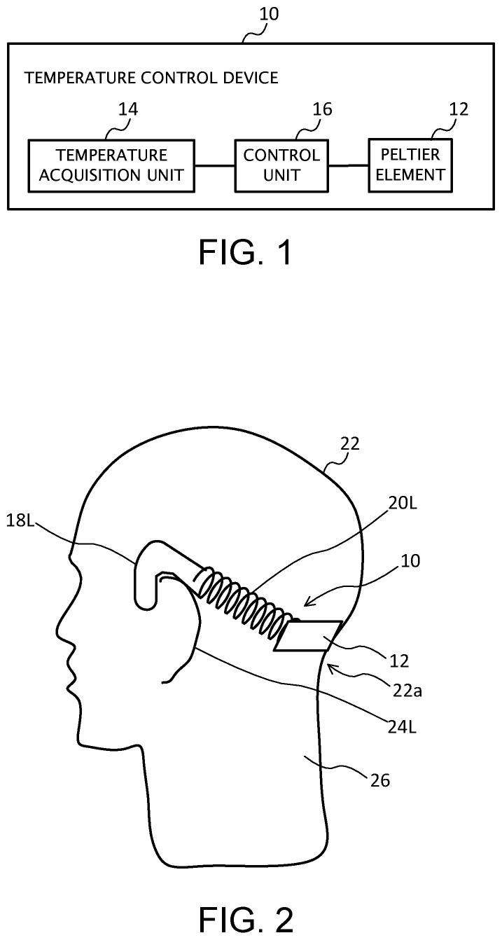

A temperature control device 10 according to an embodiment will be described with reference to FIG. 1. FIG. 1 is a block diagram illustrating a configuration of the temperature control device 10.

The temperature control device 10 includes a Peltier element 12, a temperature acquisition unit 14, and a control unit 16.

The Peltier element 12 is a thermo-electric device that provides the Peltier effect. The Peltier element 12 utilizes heat transfer and functions as a cooling device that performs cooling by absorbing heat or as a warming device that performs warming by dissipating heat. Known Peltier elements can be used as the Peltier element 12. The Peltier element 12 is one example of cooling and warming elements.

The Peltier element 12 has, for example, a flat plate shape having an upper surface and a lower surface. When a voltage is applied to make a direct current flow in a certain direction, the Peltier element 12 absorbs heat from the upper surface and dissipates heat from the lower surface. The upper surface thus functions as a cooling surface to cool the upper surface side, and the lower surface functions as a warming surface to warm the lower surface side. If the direction of the direct current is changed, the cooling surface and the warming surface are switched. The upper surface thus functions as the warming surface to warm the upper surface side, the lower surface functions as the cooling surface to cool the lower surface side.

As described below, the Peltier element 12 is worn by the user to cool or warm the user. The Peltier element 12 may be placed directly on the skin of the human user, or may be placed indirectly on the skin with clothes or the like in between. The Peltier element 12 is placed at a position corresponding to the human central nerve system on the user's skin. For example, it is placed at the center in the horizontal direction on the back of the user's head. More specifically, it is placed at a position above the back of the neck (that is, the posterior region of the neck (nape)), for example, at a position closer to the back of the head than the boundary between the back of the head and the back of the neck. The Peltier element 12 may be placed near the hairline on the back of the head. In another example, the Peltier element 12 may be placed, on the back, at a position on the spine. The central position in the horizontal direction of the back of the head or the position on the spine are positions corresponding to the central nervous system; that is, positions where the distance from the skin to the central nervous system is shorter than at other positions on the skin. By placing the Peltier element 12 at the center position in the horizontal direction of the back of the head or the position on the spine, the central nervous system can be cooled or warmed more effectively as compared to the case where the

Peltier element 12 is placed at positions other than these. This effect will be described in detail below.

The temperature acquisition unit 14 acquires information indicating the temperature around the user who wears the Peltier element 12. For example, the temperature acquisition unit 14 may acquire information indicating the temperature of a location at which the user is present via a communication path, such as the Internet; may acquire information indicating the temperature detected by a temperature sensor included in a terminal device carried or worn by the user (for example, a smartphone, smart watch, smart glasses, or other wearable devices); or may acquire information indicating the temperature detected by temperature sensors installed around the user. The temperature acquisition unit 14 may acquire information indicating a temperature included in weather information, such as a weather forecast. Information indicating the location of the user is obtained by using a known technique (for example, a Global Positioning System (GPS) function of the terminal device carried or worn by the user).

The control unit 16 controls the operations of the Peltier element 12 (for example, cooling and warming) based on the information indicating the temperature acquired by the temperature acquisition unit 14. Control of the Peltier element 12 will be described in detail below. The control unit 16 may control the operation of the Peltier device 12 based on information other than the temperature, such as, for example, heat flux and the amount of solar radiation. This means that the control unit 16 may control the operation of the Peltier element 12 based on information about the thermal environment, such as temperature, heat flux, or the amount of solar radiation. In this case, the temperature control device 10 has a function of acquiring information indicating heat flux or the amount of solar radiation.

In the temperature control device 10 described above, the configuration other than the Peltier element 12 (i.e., the temperature acquisition unit 14 and the control unit 16) can be implemented by using hardware resources, such as processors and electronic circuits. In this implementation, devices such as a memory may also be used as necessary. The configuration other than the Peltier element 12 may be implemented by using a computer. This means that all or part of the configuration other than the Peltier element 12 may be implemented by combining hardware resources, such as a Central Processing Unit (CPU) and a memory provided in the computer with software (a program) that defines the operation of the CPU and the like. The program is stored in a storage device via a recording medium, such as a CD or DVD, or via a communication path, such as a network. As an alternative example, the configuration other than the Peltier element 12 may be implemented by a Digital Signal Processor (DSP), Field Programmable Gate Array (FPGA), or the like.

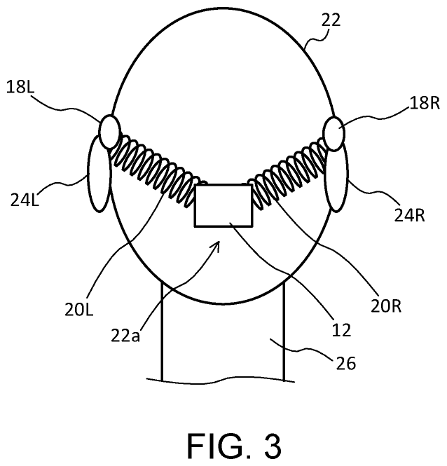

The Peltier element 12 worn by the user's head will be described with reference to FIGS. 2 and 3. FIG. 2 is a side view schematically illustrating the left side of the user's head 22. FIG. 3 is a rear view schematically illustrating the rear side of the user's head 22.

The Peltier element 12 is attached to the head 22 by ear hook portions 18R and 18L and elastic members 20R and 20L.

The ear hook portion 18R is a member to be hooked on the user's right ear 24R. The ear hook portion 18L is a member to be hooked on the user's left ear 24L.

One end of the elastic member 20R is fixed to the right end of the Peltier element 12. The other end of the elastic member 20R is fixed to the ear hook portion 18R. The elastic member 20R is provided so as to extend from the right end of the Peltier element 12 to the ear hook portion 18R. The elastic member 20R connects the Peltier element 12 and the ear hook portion 18R.

One end of the elastic member 20L is connected to the left end of the Peltier element 12. The other end of the elastic member 20L is connected to the ear hook portion 18L. The elastic member 20L is provided so as to extend from the left end of the Peltier element 12 to the ear hook portion 18L. The elastic member 20L connects the Peltier element 12 and the ear hook portion 18L.

The elastic members 20R and 20L are, for example, springs, rubber bodies, dampers, or the like. The ear hook portion 18R is hooked on the right ear 24R, and the ear hook portion 18L is hooked on the left ear 24L, so that the Peltier element 12 is placed on the back 22a of the head. The Peltier element 12 is pressed against the back 22a of the head by the tension of the elastic members 20R and 20L, thereby reducing displacement of the Peltier element 12. To place the Peltier element 12 on the back 22a of the head, the position relationships among the Peltier element 12, the ear hooks 18R and 18L, and the elastic members 20R and 20L are adjusted, and the lengths of the elastic members 20R and 20L are adjusted. The temperature control device 10 according to the embodiment has a shape like a pair of spectacles to be worn in a front/rear reverse manner, and it is therefore easy to put on and take off.

The Peltier element 12 is placed at a position that is at the center in the horizontal direction of the back of the head 22 and is closer to the back 22a of the head than to the boundary between the back 22a of the head and the posterior region of the neck 26 (the back of the neck). The Peltier element 12 is thus placed at the position corresponding to the central nerve system on the skin of the head 22. The Peltier element 12 may be placed near the hairline on the posterior region of the neck. In this case, the Peltier element 12 may be placed on the back 22a of the head with the hair in between. This makes it possible to reduce discomfort, such as extreme coldness and heat, felt by the user when the user wears the Peltier device 12. Although, in the example shown in FIGS. 2 and 3, the Peltier element 12 is placed on the back 22a of the head, it may be placed at a position other than on the back 22a of the head (for example, a position on the spine), so long as the position corresponds to the central nervous system.

Hereinafter, the skin-side surface of the Peltier element 12 will be referred to as the “lower surface,” and the opposite surface will be referred to as the “upper surface.” When the Peltier element 12 is placed directly on the skin, the surface in contact with the skin corresponds to the “lower surface.” Also, when the Peltier device 12 is placed indirectly on the skin with clothes or the like in between, the surface in contact with the clothes or the like corresponds to the “lower surface.”

The Peltier element 12, the temperature acquisition unit 14, and the control unit 16 may constitute separate modules, and the modules may constitute the temperature control device 10. Alternatively, the Peltier device 12, the temperature acquisition unit 14, and the control unit 16 may constitute a physically single module, and the module may constitute the temperature control device 10.

As an example of the separate modules, the temperature acquisition unit 14 and the control unit 16 are implemented by using terminal devices, such as a smart phone and wearable device. In this case, the Peltier element 12 is provided with a communication interface having a function of enabling short-range wireless communication, such as Bluetooth (registered trademark), and the terminal devices and the Peltier element 12 communicate with each other through the short-range wireless communication. The control unit 16 controls the operation of the Peltier element 12 through the short-range wireless communication. The terminal devices and the Peltier element 12 may be physically connected via cables, and the control unit 16 may control the operation of the Peltier element 12 through wired communication.

As an example of the single module, a processor and a memory for implementing the temperature acquisition section 14 and the control section 16 are included in the Peltier element 12, to thereby control the operation of the Peltier element 12. The processor and the memory may be included in the ear hook portions 18R and 18L.

A power supply (for example, a rechargeable battery) that supplies power to the Peltier element 12 is included in the Peltier element 12. The power supply may be included in the ear hook portions 18R and 18L rather than in the Peltier element 12 itself The Peltier element 12 is also provided with a power switch for switching the power supply in the Peltier element 12 on and off.

A temperature sensor for detecting the temperature of the upper surface is included in the upper surface of the Peltier element 12, and a temperature sensor for detecting the temperature of the lower surface is included in the lower surface of the Peltier element 12. Information indicating the temperatures detected by the temperature sensors is output to the control unit 16.

The temperature control device 10 controls the temperature perceived by the user by cooling or warming the Peltier element 12 and uses the user's own heat production, thereby enabling quick transition to an appropriate temperature.

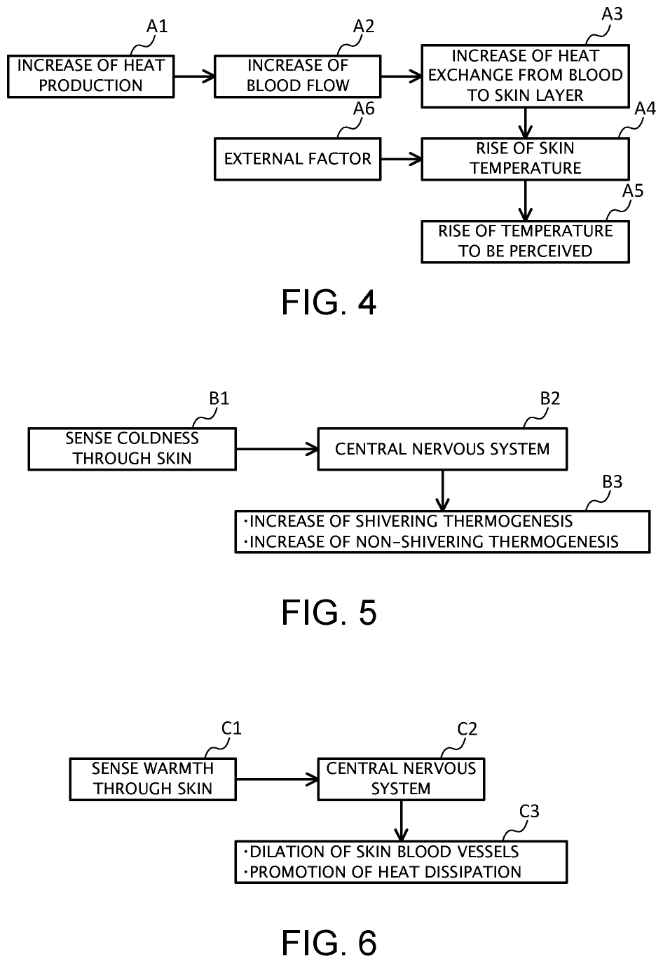

The principle of transition to the proper temperature by heat production will be described with reference to FIG. 4. FIG. 4 shows the principle to raise the temperature to be perceived.

(A1) When thermogenesis (heat production in the human body) increases due to shivering or the like, (A2) blood flow increases, and as a result, (A3) heat exchange from the blood to the skin layer increases. This causes (A4) the skin temperature to rise, and as a result, (A5) the temperature to be perceived rises. (A6) The skin temperature also rises due to external factors, such as heat conduction, convection, and radiation.

In the present embodiment, (Al) heat production and heat dissipation are used to raise or lower the temperature perceived by the user. By using heat production and heat dissipation, (A5) the temperature to be perceived can be raised or lowered with less electric power than when the skin temperature is raised or lowered by external factors.

FIG. 5 shows a mechanism to increase heat production.

(B1) When a person's skin is cooled, and the person senses coldness, (B2) the coldness is transmitted to the central nervous system. As a result, (B3) a command of “shivering” is transmitted to the skeletal muscle via the preoptic area, the hypothalamus, the motor nerves, etc., and the skeletal muscle that receives the command shivers. This results in “shivering thermogenesis.” In addition, activation of sympathetic nerves transmits a command of heat production to brown adipose tissue and the like, and “non-shivering thermogenesis” occurs in the brown adipose tissue and the like. As a result, the skin temperature rises, and the temperature perceived by the person rises. Even if the skin temperature is not changed, the lowered temperature of the central nervous system causes “shivering thermogenesis” and “non-shivering thermogenesis,” resulting in increased thermogenesis. In the present embodiment, the Peltier element 12 is placed at the position corresponding to the central nervous system, and the temperature of the central nervous system can thus be effectively lowered as compared to the case where the Peltier element 12 is placed at other positions. In other words, the Peltier element 12 cools the skin on which it is placed, and the central nervous system thus detects, as the temperature perceived by the person, the coldness that is enhanced as compared to that in the case without the Peltier element 12. Transition to the appropriate temperature is then performed according to the enhanced coldness. The transition can therefore be performed more quickly.

FIG. 6 shows a mechanism to increase heat dissipation. (C1) When a person's skin is warmed, and the person senses warmth, (C2) the warmth is transmitted to the central nervous system. As a result, (C3) a command of “dilation” is transmitted to the skin blood vessels via the preoptic area, the hypothalamus, the sympathetic ganglia, and the like, and the skin blood vessels dilate to promote heat dissipation. Consequently, the skin temperature falls, and the temperature perceived by the person falls. In other words, the Peltier element 12 warms the skin on which it is placed, and the central nervous system thus detects, as the temperature perceived by the person, the warmth that is enhanced as compared to that in the case without the Peltier element 12. Transition to the appropriate temperature is then performed according to the enhanced warmth. The transition can therefore be performed more quickly.

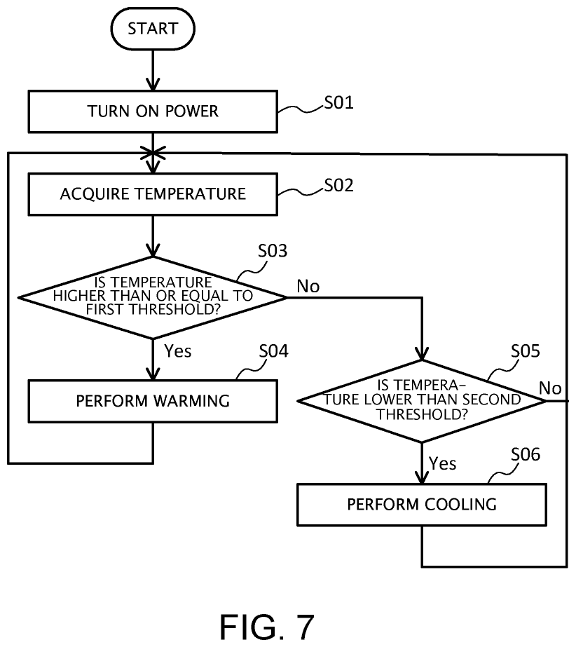

Hereinafter, the operations of the temperature control device 10 will be described with reference to FIG. 7.

First, the Peltier element 12 is powered by turning on the power switch on the Peltier element 12 (S01). Although, here, the Peltier device 12 is powered on by way of an example, the temperature control device 10 may be powered on. The temperature control device 10 is powered on to thereby supply power to the Peltier element 12.

The temperature acquisition unit 14 acquires information indicating the temperature around the user (S02). The acquired temperature information is output to the control unit 16.

When the acquired temperature is higher than or equal to a first threshold (Yes in S03), the control unit 16 controls the operation of the Peltier element 12 to warm the user's skin (S04). Specifically, the control unit 16 controls the operation of the Peltier element 12 such that the lower surface of the Peltier element 12 dissipates heat and functions as the warming surface. This warms the skin, and the temperature to be perceived falls. The procedure proceeds to step S02.

When the acquired temperature is not higher than or equal to the first threshold (No in S03), the procedure then proceeds to step S05.

When the acquired temperature is lower than a second threshold that is lower than the first threshold (Yes in S05), the control unit 16 controls the operation of the Peltier element 12 to cool the user's skin (S06). Specifically, the control unit 16 controls the operation of the Peltier element 12 such that the lower surface of the Peltier element 12 absorbs heat and functions as the cooling surface. This cools the skin, and the temperature to be perceived rises. The procedure proceeds to step S02.

When the acquired temperature is higher than or equal to the second threshold (No in 505), the procedure then proceeds to step S02.

The first threshold and the second threshold are predetermined values. The first threshold and the second threshold may be set by the user.

The first threshold is set such that the lower surface of the Peltier element 12 functions as the warming surface when it is assumed that the user feels hot. Thus, when the user feels hot, the lower surface of the Peltier element 12 functions as the warming surface, and warms the user's skin, to thereby lower the temperature perceived by the user.

The second threshold is set such that the lower surface of the Peltier element 12 functions as the cooling surface when it is assumed that the user feels cold. Thus, when the user feels cold, the lower surface of the Peltier element 12 functions as the cooling surface, and cools the user's skin, to thereby raise the temperature perceived by the user.

As such, when it is cold around the user, the temperature control device 10 cools the user's skin to enhance the coldness of the temperature perceived by the user. This results in heat production by the user, and produced heat is used to raise the temperature. On the other hand, when it is hot around the user, the temperature control unit 10 warms the user's skin to enhance the warmth of the temperature perceived by the user. This results in heat dissipation by the user to thereby decrease the temperature. It is thus possible to effectively control the temperature perceived by the user.

In the above embodiment, cooling and warming are automatically switched, but the user may switch between cooling and warming. For example, the Peltier element 12 is provided with a switch for switching between cooling and warming modes. When the user switches the switch to the cooling mode, the control unit 16 causes the lower surface of the Peltier element 12 to function as the cooling surface. When the user switches the switch to the warming mode, the control unit 16 causes the lower surface of the Peltier element 12 to function as the warming surface.

It is also possible to provide signs “cold” and “hot” on the switch. When the user switches the switch to “cold,” the control unit 16 then causes the lower surface of the Peltier element 12 to function as the cooling surface. When the user switches the switch to “hot,” the control unit 16 then causes the lower surface of the Peltier element 12 to function as the warming surface.

A modified example will now be described.

When the Peltier element 12, for example, performs cooling (that is, when its lower surface functions as the cooling surface), it becomes difficult to lower the temperature of the lower surface as the temperature of the upper surface, which is the heat dissipation surface, rises. It may thus become difficult for the user to feel stimulation from the temperature. A similar situation occurs in the case of warming (that is, when the lower surface functions as the warming surface).

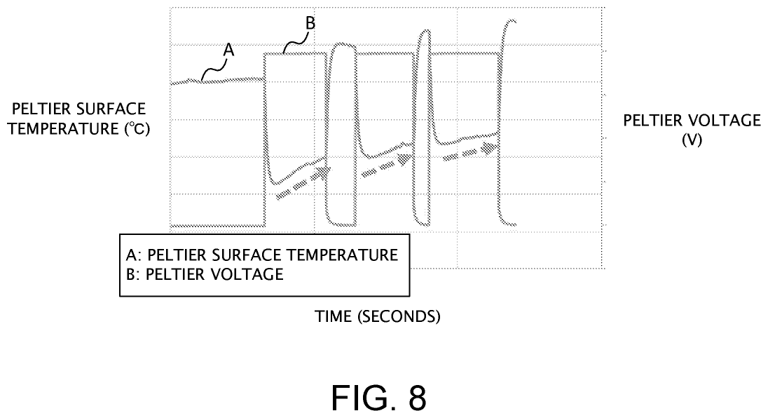

FIG. 8 shows changes in temperature of the cooling surface of the Peltier element over time. In FIG. 8, the horizontal axis indicates the time that has elapsed since the Peltier element was powered on. The vertical axis on the left side indicates a surface temperature of the Peltier element. The vertical axis on the right side indicates a voltage applied to the Peltier element. A reference sign A indicates changes in surface temperature of the Peltier element over time. A reference sign B indicates changes in voltage applied to the Peltier element over time.

In the example shown in FIG. 8, the Peltier element is switched on and off repeatedly at predetermined time intervals.

The situation where the Peltier element is on during cooling means that the Peltier element is powered on, and that a voltage is applied to the Peltier element so that the lower surface of the Peltier element functions as the cooling surface. The situation where the

Peltier element is off during cooling means that the Peltier element is powered off, the Peltier element is powered on but no voltage is applied to the Peltier element, or that the Peltier element is powered on and a voltage that is too small for the lower surface to function as the cooling surface is applied. Although, here, the Peltier element is switched on and off by way of an example, the temperature control device 10 may be switched on and off.

The situation where the Peltier element is on during warming means that the Peltier element is powered on, and that a voltage is applied to the Peltier element so that the lower surface of the Peltier element functions as the warming surface. The situation where the Peltier element is off during warming means that the Peltier element is powered off, the Peltier element is powered on but no voltage is applied to the Peltier element, or that the Peltier element is powered on and a voltage that is too small for the lower surface to function as the warming surface is applied.

Due to characteristics of the Peltier element, if its back surface (corresponding to the upper surface in this embodiment) has poor heat dissipation and heat absorption performance, the degree of cooling and warming by its front surface (corresponding to the lower surface in this embodiment) is reduced. As shown in FIG. 8, even if the voltages of the same magnitude are applied to the Peltier element, the longer the time elapsed since the Peltier element is powered on, the smaller the degree of surface temperature fall during cooling becomes, resulting in difficulty in lowering the surface temperature.

Therefore, the control unit 16 switches on and off the Peltier device 12 repeatedly, in order to prevent temperature rise of the upper surface during cooling and prevent temperature drop of the upper surface during warming. For example, the control unit 16 switches between applying and removing voltage to the Peltier device 12 in a repeated manner. That is, the control unit 16 switches between application of voltage (voltage ON) and non-application of voltage (voltage OFF) to the Peltier element 12 in a repeated manner.

In addition, during cooling (that is, when the lower surface functions as the cooling surface), when the temperature of the lower surface of the Peltier element 12 does not fall below a third threshold, the control unit 16 makes the length of time during which the Peltier element 12 is off longer than those in other cases.

Similarly, during warming (that is, when the lower surface functions as the warming surface), when the temperature of the lower surface of the Peltier element 12 does not rise above a fourth threshold, the control unit 16 makes the length of time during which the Peltier element 12 is off longer than those in other cases.

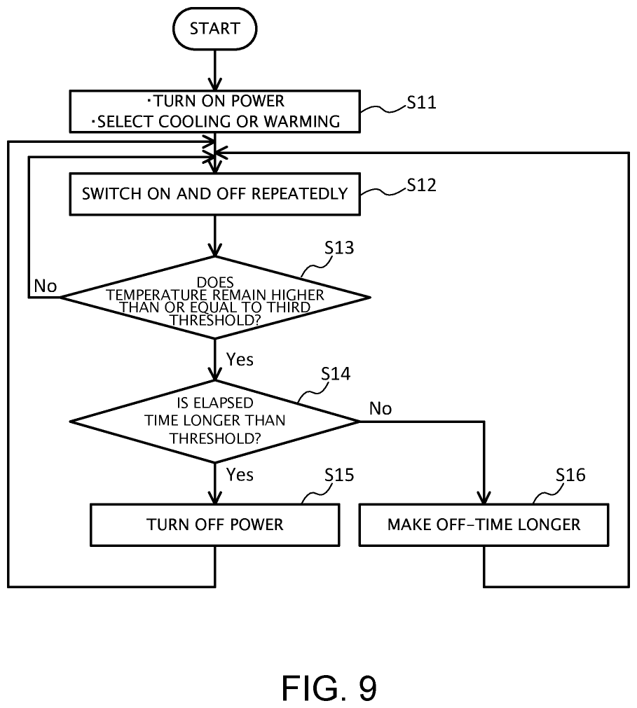

The operations according to the modified example will be described with reference to FIG. 9. FIG. 9 is a flowchart illustrating operations according to the modified example. Here, by way of an example, it is assumed that the user selects cooling or warming.

First, the user turns on the Peltier device 12 and selects cooling or warming (S11). For example, the user selects cooling or warming by switching a switch for switching between cooling and warming modes. When the cooling mode is selected, the lower surface of the Peltier element 12 is cooled. When the warming mode is selected, the lower surface of the Peltier element 12 is warmed. Here, by way of an example, it is assumed that the user selects the cooling mode.

The control unit 16 switches on and off the Peltier element 12 repeatedly at predetermined switching time intervals (S12). For example, the length of time during which the element is on is four minutes, and the length of time during which the element is off is one minute. This is merely an example, and the length of the time periods may be changed by the user. The length of on-time and the length of off-time may be equal.

When the temperature of the lower surface of the Peltier element 12 falls below the third threshold (No in S13), the procedure returns to step S12.

When the temperature of the lower surface of the Peltier element 12 does not fall below the third threshold (Yes in S13), the procedure proceeds to step S14.

For example, the third threshold is set based on an initial temperature (for example, the temperature of the lower surface at the time when the Peltier device 12 is powered on).

Specifically, (initial temperature—X [° C.]) is set as the third threshold. X [° C.] is a predetermined value. X [° C.] may be set by the user.

As a specific example, if X [° C.] is 5 [° C.], and the initial temperature is 25 [° C.], the third threshold is then 20 [° C.]. When the temperature of the lower surface of Peltier element 12 does not fall below 20 [° C.], the procedure proceeds to step S14. If not, that is, when the temperature of the lower surface of the Peltier element 12 falls below 20[° C.], the procedure proceeds to step S12. The above temperature values are merely examples.

The processes in step S14 and subsequent steps will now be described.

When the elapsed time since the Peltier element 12 is powered on is longer than or equal to a threshold (Yes in S14), the control unit 16 then powers off the Peltier element 12

(S15). The operation of the Peltier element 12 is thus stopped, so that the temperature of the warmed upper surface of the Peltier element 12 decreases, and the temperature of the lower surface of the Peltier element 12 becomes easy to decrease after the Peltier element 12 is powered on again. The control unit 16 powers on the Peltier element 12 again after a predetermined time period has passed after the Peltier element 12 was powered off. Then, the procedure proceeds to step S12.

When the elapsed time since the Peltier element 12 is powered on is shorter than the threshold (No in S14), the control unit 16 then makes the length of time during which the element is off longer when switching the Peltier element 12 between on and off (S16). For example, if the length of time during which the Peltier element 12 is off is one minute, the control unit 16 extends this length to two minutes. As the length of off-time becomes greater, the temperature of the heated upper surface of the Peltier element 12 decreases, and the temperature of the lower surface of the Peltier element 12 becomes easy to decrease. The procedure then proceeds to step S12. When the temperature of the lower surface falls below the third threshold (No in S13), the control unit 16 restores the length of the off-time to the original length (for example, one minute). The length of time is merely an example and may be changed by the user.

As described above, by powering off the Peltier element 12 when the temperature of the lower surface of the Peltier element 12 does not fall below the third threshold value, the temperature of the upper surface becomes easier to decrease, and the temperature of the lower surface thus becomes easier to decrease after the Peltier element 12 is powered on again compared to when the Peltier element 12 is continuously powered without being turned off. In addition, by making the length of time during which the Peltier element 12 is off longer, the temperature of the upper surface of the Peltier element 12 becomes easier to decrease, and the temperature of the lower surface of the Peltier element 12 thus becomes easier to decrease compared to when the length of the off-time is not changed.

During warming, the Peltier element 12 is also controlled to be switched between on and off in the same manner as during cooling. For example, during warming, when the temperature of the lower surface of the Peltier element 12 does not rise above the fourth threshold, the processes in step S14 and subsequent steps are performed.

For example, the fourth threshold is set based on the initial temperature. Specifically, (initial temperature +Y [° C.]) is set as the fourth threshold. Y [° C.] is a predetermined value. Y [° C.] may be set by the user.

As a specific example, if Y [° C.] is 5 [° C.], and the initial temperature is 25 [° C.], the fourth threshold is then 30 [° C.]. When the temperature of the lower surface of Peltier element 12 does not rise above 30[° C.], the procedure proceeds to step S14. If not, that is, when the temperature of the lower surface of the Peltier element 12 rises above 30[° C.], the procedure proceeds to step S12. The processes in step S14 and subsequent steps are the same as those described above. The temperature of the lower surface of the Peltier element 12 thus becomes easy to rise. The above temperature values are merely examples.

Although, in the embodiment, the example in which the Peltier element 12 is attached to a person has been described, it may be attached to an animal other than a person to perform the operations according to the embodiment.

1. A temperature control device comprising:
an acquisition unit that acquires information indicating a temperature around a user;
a cooling and warming element that is worn by the user to cool or warm the user; and
a control unit that controls the cooling and warming element based on the temperature acquired by the acquisition unit, wherein
the control unit causes the cooling and warming element to warm the user when the temperature is higher than or equal to a first threshold and causes the cooling and warming element to cool the user when the temperature is lower than a second threshold that is lower than the first threshold.

2. The temperature control device according to claim 1, wherein the cooling and warming element is placed on a back of a head of the user.

3. The temperature control device according to claim 1, wherein the cooling and warming element is placed at a position corresponding to a central nerve system of the user.

4. The temperature control device according to claim 2, wherein the cooling and warming element is placed at a position corresponding to a central nerve system of the user.

5. The temperature control device according to claim 1, wherein
the control unit switches on and off the cooling and warming element repeatedly, and
when a temperature of a user-facing surface of the cooling and warming element does not fall below a third threshold while the cooling and warming element performs cooling, or when the temperature of the user-facing surface of the cooling and warming element does not rise above a fourth threshold while the cooling and warming element performs warming, the control unit makes the length of time during which the cooling and warming element is off longer than those in other cases.

6. The temperature control device according to claim 2, wherein
the control unit switches on and off the cooling and warming element repeatedly, and
when a temperature of a user-facing surface of the cooling and warming element does not fall below a third threshold while the cooling and warming element performs cooling, or when the temperature of the user-facing surface of the cooling and warming element does not rise above a fourth threshold while the cooling and warming element performs warming, the control unit makes the length of time during which the cooling and warming element is off longer than those in other cases.

3. emperature control device according to claim 3, wherein
the control unit switches on and off the cooling and warming element repeatedly, and
when a temperature of a user-facing surface of the cooling and warming element does not fall below a third threshold while the cooling and warming element performs cooling, or when the temperature of the user-facing surface of the cooling and warming element does not rise above a fourth threshold while the cooling and warming element performs warming, the control unit makes the length of time during which the cooling and warming element is off longer than those in other cases.

8. The temperature control device according to claim 4, wherein
the control unit switches on and off the cooling and warming element repeatedly, and
when a temperature of a user-facing surface of the cooling and warming element does not fall below a third threshold while the cooling and warming element performs cooling, or when the temperature of the user-facing surface of the cooling and warming element does not rise above a fourth threshold while the cooling and warming element performs warming, the control unit makes the length of time during which the cooling and warming element is off longer than those in other cases.

9. The temperature control device according to claim 1, wherein the cooling and warming element is a Peltier element.

10. The temperature control device according to claim 2, wherein the cooling and warming element is a Peltier element.

11. The temperature control device according to claim 3, wherein the cooling and warming element is a Peltier element.

12. The temperature control device according to claim 4, wherein the cooling and warming element is a Peltier element.

13. The temperature control device according to claim 5, wherein the cooling and warming element is a Peltier element.
